(19) United States
(12) Patent Application Publication
(10) Pub. No.: US 2015/0216874 A1
(43) Pub. Date: Aug. 6, 2015

(54) ANTIBIOTIC COMPOSITIONS AND METHODS

(71) Applicant: Roy Martin, Downers Grove, IL (US)

(72) Inventor: Roy Martin, Downers Grove, IL (US)

(73) Assignee: Truox, Inc., McClellan, CA (US)

(21) Appl. No.: 14/544,441

(22) Filed: Jan. 7, 2015

Related U.S. Application Data
(60) Provisional application No. 61/965,757, filed on Feb. 6, 2014.

(51) Int. Cl. A61K 31/545; A61K 31/407

(52) U.S. Cl. CPC A61K 31/545; A61K 31/407

(57) ABSTRACT

Disclosed is a novel family of antibiotic that provides bacteria specific targeting, activation and the ability to prevent bacteria mutations that result in bacteria resistance. The compositions and methods of the invention provide for an antibiotic that is effective against Methicillin-resistant Staphylococcus aureus (MRSA).

### FIELD OF INVENTION

The present invention relates to antibiotic compositions and methods for treating antibiotic resistant bacteria and reducing the potential for bacteria mutations that resist the antibiotic efficacy of the antibiotic compositions comprising the invention.

### BACKGROUND

β-Lactam based antibiotics target specifically the cell wall found only in bacteria. This attributes to their relative low toxicity. The cell wall of bacteria is constructed by the enzyme DD-transpeptidase. The cell-wall structure prevents osmotic pressure from causing cell lysis and subsequent death. β-Lactam based antibiotics attack the serine site of the DD-transpeptidase, covalently bond to it, and effectively compromises the enzymes ability to function in the construction of the cell wall. This process carried out extensively within the cell wall compromises the cell wall integrity, which leads to lysis of the cell.

The β-Lactam family of antibiotics has gone through several generations of evolution. The general structures of each generation are illustrated.

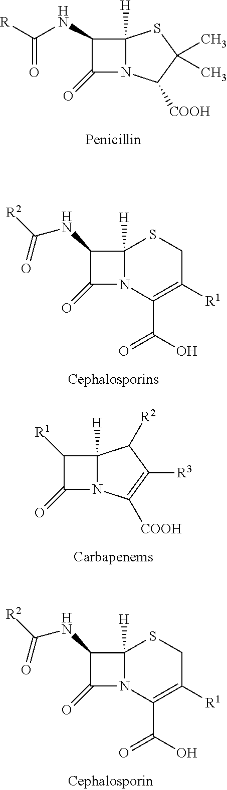

Referring to the “R¹” group in the Cephalosporin illustration, the first generation of Cephalosporins comprised hydrogen, methyl and oxymethyl groups. It was determined that the β-Lactam ring of the cephalosporins was not as reactive compared to penicillin. To improve the reactivity of the β-Lactam ring, a chlorine group was substituted at the “R¹” site. This was found to increase the electronegative value which facilitates opening of the β-Lactam ring.

Newer generations of cephalosporins incorporate leaving groups that further increase the reactivity of the β-Lactam ring.

The β-lactam antibiotics are useful and frequently prescribed antimicrobial agents that share a common structure and mechanism of action: inhibition of synthesis of the bacterial peptidoglycan cell wall. The group includes penicillins, cephalosporins, and carbapenems. The penicillins consist of penicillins G and V, which are highly active against susceptible gram-positive cocci; penicillinase-resistant penicillins such as nafcillin, which are active against penicillinase-producing Staphylococcus aureus; ampicillin and other agents with an improved gram-negative spectrum, especially when combined with β-lactamase inhibitor; and extended-spectrum penicillins with activity against Pseudomonas aeruginosa, such as piperacillin.

The β-lactams also include the cephalosporin antibiotics, which are classified by generation: First-generation agents have excellent gram-positive and modest gram-negative activity; second-generation agents have somewhat better activity against gram-negative organisms and include some agents with anti-anaerobe activity; third-generation agents have activity against gram-positive organisms and much more activity against the Enterobacteriaceae, with a subset active against P. aeruginosa; and fourth-generation agents encompass the antimicrobial spectrum of all the third-generation agents and have increased stability to hydrolysis by inducible chromosomal β-lactamases.

Carbapenems, including imipenem, doripenem, ertapenem and meropenem, have the broadest antimicrobial spectrum of any antibiotic, whereas the monobactam aztreonam has a gram-negative spectrum resembling that of the aminoglycosides.

β-Lactamase inhibitors such as clavulanate are used to extend the spectrum of penicillins against β-lactamase-producing organisms. Bacterial resistance against the β-lactam antibiotics continues to increase at a dramatic rate. Mechanisms of resistance include not only production of β-lactamases that in some cases destroy all β-lactam antibiotics, but also alterations in or acquisition of novel penicillin-binding proteins PBPs and decreased entry and/or active efflux of the antibiotic. It is not an exaggeration to state that we are re-entering the pre-antibiotic era, with many nosocomially acquired gram-negative bacterial infections resistant to all available antibiotics.

The need for antibiotics that provide biocide efficacy against antibiotic resistant bacteria commonly referred to as “Methicillin-resistant Staphylococcus aureus (MRSA)” is critical.

### DEFINITIONS

As used herein, “carbon based backbone” is the organic portion of the organic acyl polyoxychlorine compound and includes the atom directly appending the acyl carbon of the acyl polyoxychlorine group. The carbon based backbone is represented by “R” in the following general formula:

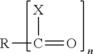

Non-limiting examples of the carbon based backbone comprise: alkyl, aryl, aryl alkyl, cycloalkyl, carbocyclic, and heterocyclic groups. The carbon based backbone can be saturated or unsaturated as well as substituted or unsubstituted. The carbon based backbone may be substituted with moiety exemplified by the non-limiting examples: nitrogen, sulfur, phosphorous, oxygen, carboxyl, alkoxy, hydroxyl, carbonyl, chlorine, fluorine, and the like. Substituted moiety may be selected to further increase the electronegative properties of the OAP leaving group.

As used herein, “bacteria activated Beta-Lactam carrier” also referred to as “β-Lactam carrier” consist of a compound comprising a Beta-lactam group (also referred to as “Beta-Lactam ring”) that reacts with enzymes located within the peptidoglycan cell wall of bacteria. The β-Lactam carrier is coupled (covalently bonded) to organic acyl polyoxychlorine (OAP) to form the OAP based antibiotic compositions of the invention. The preferred β-Lactam carrier comprises cephalosporin, but is not limited to cephalosporin. The β-Lactam carrier may be any bacteria activated Beta-Lactam carrier that releases at least one: OAP leaving group and/or an oxychlorine intermediate when the Beta-Lactam ring is cleaved by enzymatic attack within the peptidoglycan cell wall of bacteria.

As used herein, “OAP leaving group” also referred to as “leaving group” and “release group” comprises an organic acyl polyoxychlorine (OAP) compound released by the OAP based antibiotic when the β-Lactam group is reacted with enzymes within the peptidoglycan cell wall of bacteria.

As used herein, “OAP based antibiotic” also referred to as “organic acyl polyoxychlorine based antibiotic” comprises a β-Lactam carrier coupled to an organic acyl polyoxychlorine compound that kills bacteria.

As used herein, “atom directly appending the acyl carbon” describes the atom of the carbon based backbone coupled to the acyl carbon of the acyl polyoxychlorine group. Non-limiting examples of atoms directly appending the acyl carbon may include: carbon, nitrogen, sulfur, oxygen and phosphorous.

As used herein, “polyoxychlorine” describes the source of oxychlorine intermediates coupled to the organic acyl donor resulting in the formation of the organic acyl polyoxychlorine. The polyoxychlorine is selected from at least one chlorate having the general formula ClO₃ and perchlorate having the general formula ClO₄ and is coupled to the acyl carbon of the organic acyl polyoxychlorine.

As used herein, “polyoxychlorine donor” includes sources the anions of chlorate having the general formula ClO₃⁻ and anions of perchlorate having the general formula ClO₄⁻. Non-limiting examples of polyoxychlorine donors include: perchloric acid, sodium perchlorate, potassium perchlorate, lithium perchlorate, calcium perchlorate, magnesium perchlorate, sodium chlorate, potassium chlorate, lithium chlorate, calcium chlorate, magnesium chlorate and the like.

As used herein, “acyl polyoxychlorine group” is represented by the bracketed portion in the general formula:

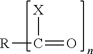

The acyl polyoxychlorine group comprises the acyl carbon, double bonded oxygen, and the polyoxychlorine represented by “X”.

As used herein, “organic acyl polyoxychlorine” also referred to as “OAP” is an organic compound comprising an acyl polyoxychlorine group, whereby reduction of the acyl polyoxychlorine group results in oxychlorine intermediates. At least one of the oxychlorine intermediates is chlorine dioxide. The reduction of the organic acyl polyoxychlorine within the bacteria induces oxidative stress and cell death.

As used herein, “oxychlorine intermediate” comprises the oxychlorine compounds resulting from the initial reduction of the acyl polyoxychlorine group and the subsequent oxychlorine compounds resulting from the reduction of the initial oxychlorine intermediate. The oxychlorine intermediates comprise the chlorine atom bound to either: one, two, three or four (in the case of perchlorate) oxygens and their various radical and ionic species resulting from the reduction of the acyl polyoxychlorine. In one non-limiting example, when an acyl polyoxychlorine group resulting from reaction of a chlorate anion with an organic acyl donor undergoes chemical reduction thereby releasing a carboxyl group and the oxychlorine intermediate chlorine dioxide, the resulting oxychlorine intermediates comprise chlorine dioxide followed by subsequent oxychlorine species exemplified by chlorite. In another non-limiting example, when the acyl polyoxychlorine undergoes enzymatic attack (e.g. acetyl coenzyme A), an oxychlorine intermediate comprising an unstable chlorate radical is released. The said radical then undergoes cascading decomposition resulting in a series of oxychlorine intermediates exemplified by chlorine dioxide.

As used herein, “coupled” is used to describe the chemical bonding that links atoms and/or groups. For example, the OAP leaving group is coupled to the Beta-Lactam carrier.

As used herein, “antibiotic composition” comprises at least an OAP based antibiotic. The antibiotic composition may further include a pharmaceutically acceptable carrier.

As used herein, the singular forms “a,” “an,” and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

### SUMMARY OF THE INVENTION

OAP based antibiotics comprise an organic acyl polyoxychlorine group having the general formula:

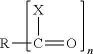

wherein (X) comprises a polyoxychlorine;
(R) comprises a carbon based backbone coupled to an atom directly appending the acyl carbon;

where (n) is one or more.

The organic acyl polyoxychlorine can be produced by reaction between an organic acyl donor and polyoxychlorine donor.

The organic acyl donors may be selected from carboxylic acids and derivatives of carboxylic acids, as well as in some cases aldehydes. Preferred examples of carboxylic acid derivatives include organic acid chlorides and organic acid anhydrides. Carboxylic acids, esters, amides and potentially aldehydes may be converted to organic acyl polyoxychlorine under suitable conditions.

The conditions necessary to produce the desired organic acyl polyoxychlorine can vary significantly dependent on the source of organic acyl donor. For example, acyl chlorides are very susceptible to nucleophilic acyl substitution when combined with polyoxychlorine anion donors. Acid anhydrides are also very useful and are readily available in both solid and liquid forms. These reactive carboxylic acid derivatives allow for broader range of reaction conditions while resulting in efficient conversion of reactants to the desired organic acyl polyoxychlorine compounds.

Carboxylic acids, esters and amides may also be used to produce the organic acyl polyoxychlorine compositions of the invention but in general require their own set of conditions (e.g. higher temperatures, pH) and/or longer reaction times when compared to acid anhydrides and acid chlorides.

Solvents should be selected to maximize conversion into the organic acyl polyoxychlorine. Non-limiting examples of suitable solvents may include: water, dimethylformamide (DMF) and dimethylsulfoxide (DMSO).

Techniques for purification exemplified by solvent extraction, membrane separation, crystallization and the like may be used to separate the organic acyl polyoxychlorine from reactants and undesirable by products before reacting with the cephalosporin carrier to produce the OAP based antibiotic.

### OAP Based Antibiotics

The novel antibiotics will be described herein with reference to novel theories and mechanisms. The claimed invention is not limited to the novel theories and mechanisms described herein.

Due to the chemical structure of organic acyl polyoxychlorine (OAP), a high level of inherent stability exist due to the steric hindrance provided by the crowded electron field (shadowed region illustrated below) surrounding the potential activation sites centered at the chlorine atom. The chlorine centered functionality is drawn to the electrophilic acyl carbon. This is believed to induce an umbrella like electron cloud, capped by the two double bonded oxygens.

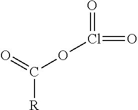

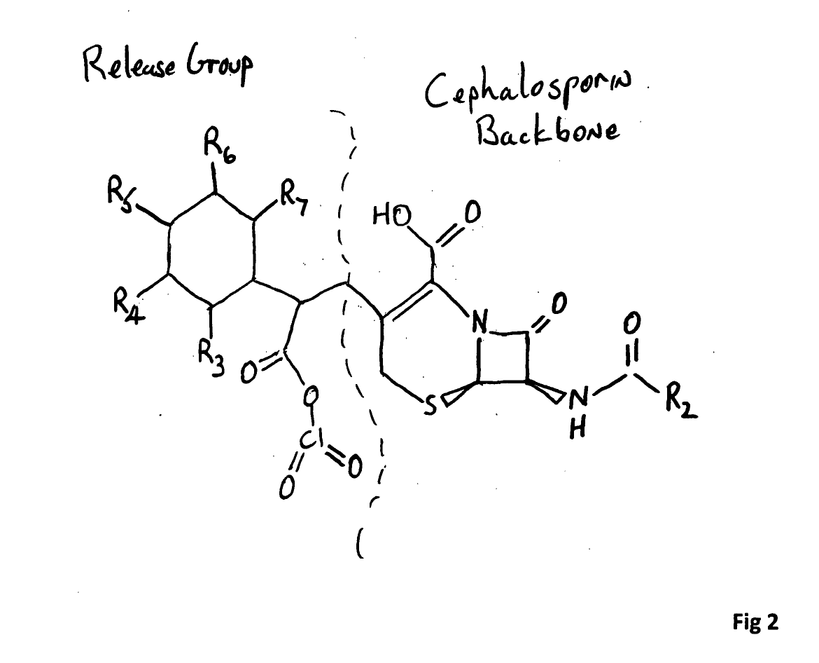

Furthermore, steric hindrance is structured into the design of the OAP based antibiotic as exemplified by the following non-limiting illustration (FIG. 2):

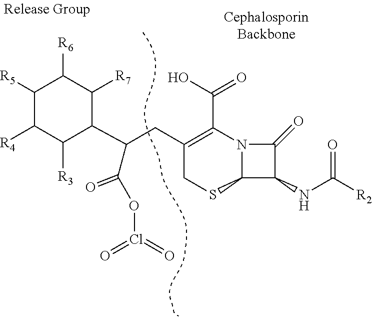

As exemplified by the non-limiting example, the cephalosporin carrier used to transport the OAP into the wall of the bacterial cell as well as trigger the release of the OAP, increases the steric hindrance and subsequent protection from premature activation of the polyoxychlorine group. The carbon based backbone can be selected to provide additional steric hindrance as well as improve the transport of the OAP leaving group (release group) through the plasma membrane of the bacteria once the β-lactam group is activated and the OAP is released. In the non-limiting illustration, R₃ may comprise an alkyl or aryl group to further increase the steric hindrance and subsequent survivability of the polyoxychlorine group during transport through the metabolic pathways of a mammal (e.g. humans, cattle etc.).

In one embodiment of the invention, there is provided an OAP based antibiotic comprising: an OAP leaving group coupled to a β-Lactam carrier.

In another embodiment of the invention, there is provided an antibiotic composition comprising: an OAP based antibiotic and a pharmaceutically acceptable carrier.

In another embodiment of the invention, the OAP based antibiotic effectively kills Methicillin-resistant Staphylococcus aureus (MRSA).

In another embodiment of the invention, there is provided a method for killing bacteria comprising the steps of:

reacting a polyoxychlorine donor with an organic acyl donor to form an OAP leaving group; coupling the OAP leaving group and Beta-lactam carrier to form an OAP based antibiotic; combining an OAP based antibiotic and a pharmaceutically acceptable carrier to form an antibiotic composition; administering said antibiotic composition to a mammal so that at least the OAP based antibiotic is absorbed into the peptidoglycan cell wall of bacteria; enzyme activation of the Beta-Lactam group; release of the OAP leaving group, and reduction of the organic acyl polyoxychlorine releases oxychlorine intermediates that induce oxidative stress and cell death.

In another embodiment of the invention, there is provided a method for killing bacteria comprising the steps of:

reacting a polyoxychlorine donor with an organic acyl donor to form an OAP leaving group; coupling the OAP leaving group and Beta-lactam carrier to form an OAP based antibiotic; combining an OAP based antibiotic and a pharmaceutically acceptable carrier to form an antibiotic composition; administering said antibiotic composition to a mammal so that at least the OAP based antibiotic is absorbed into the peptidoglycan cell wall of bacteria; enzyme activation of the Beta-Lactam group; release of oxychlorine intermediates, and reduction of the oxychlorine intermediates induces oxidative stress and cell death.

### DETAILED DESCRIPTION OF THE INVENTION

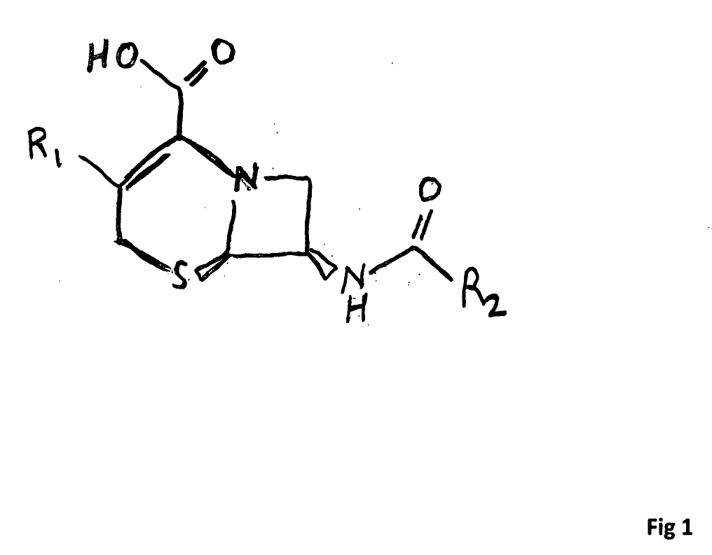

FIG. 1 illustrates the basic structure of the cephalosporin wherein the location designed “R₁” is the site where the OAP leaving group is coupled to the cephalosporin (Beta-Lactam) carrier.

FIG. 2 illustrates the basic structure represented by one non-limiting example comprising an OAP leaving group coupled to the cephalosporin carrier to form the OAP based antibiotic. Groups R₃ thru R₇ may represent hydrogen, alkyl and aryl groups.

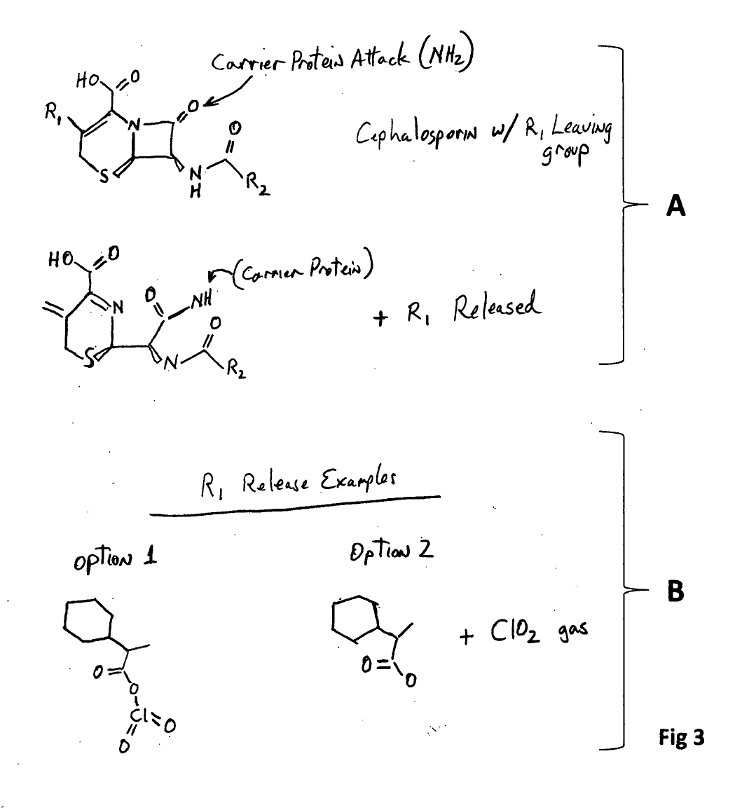

FIG. 3 illustrates the site of attack of the β-lactam ring, the cleaving (opening of the ring), and the subsequent release of the OAP leaving group (R₁). The figure also provides two non-limiting examples of potential products resulting from the released OAP leaving group.

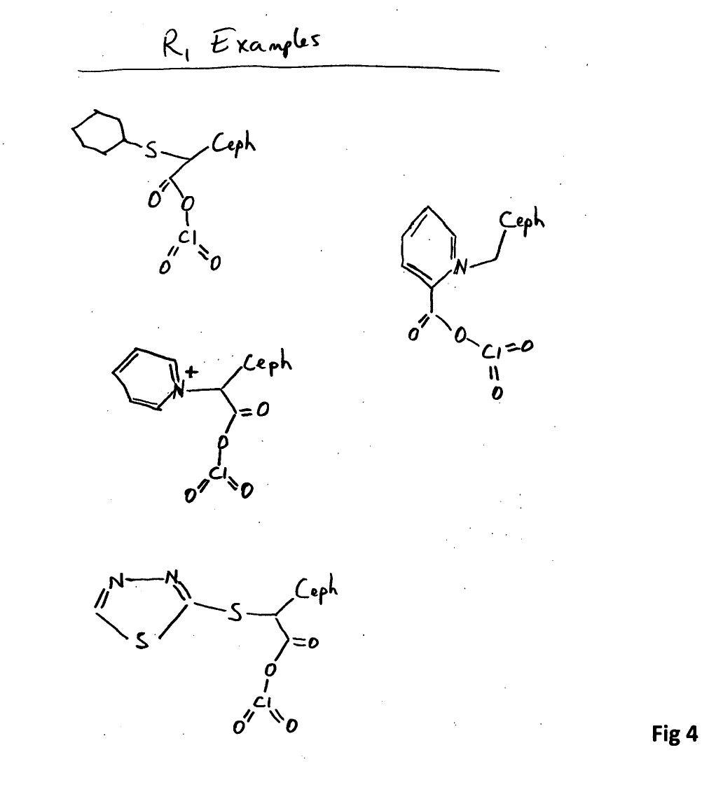

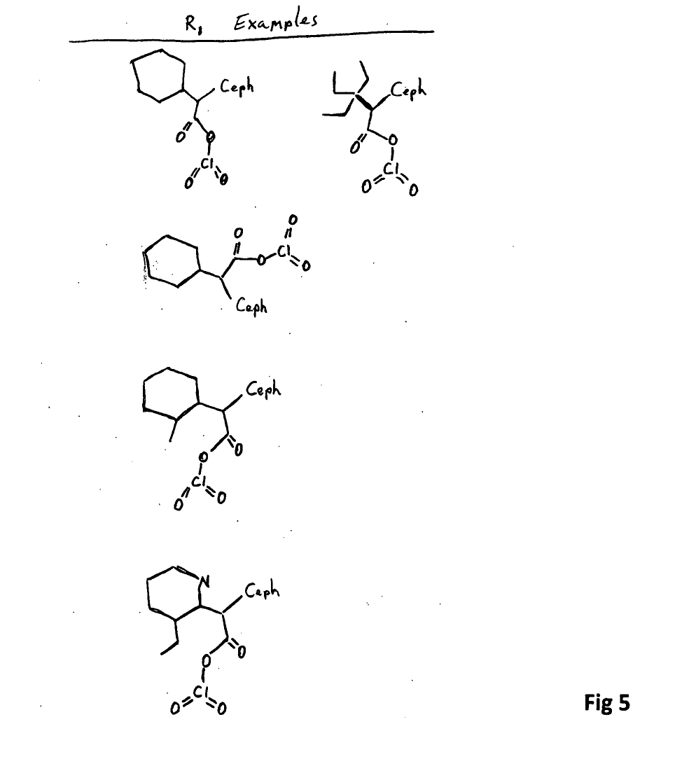

FIG. 4 and FIG. 5 illustrate non-limiting examples of OAP leaving groups coupled to the cephalosporin (designated “ceph”).

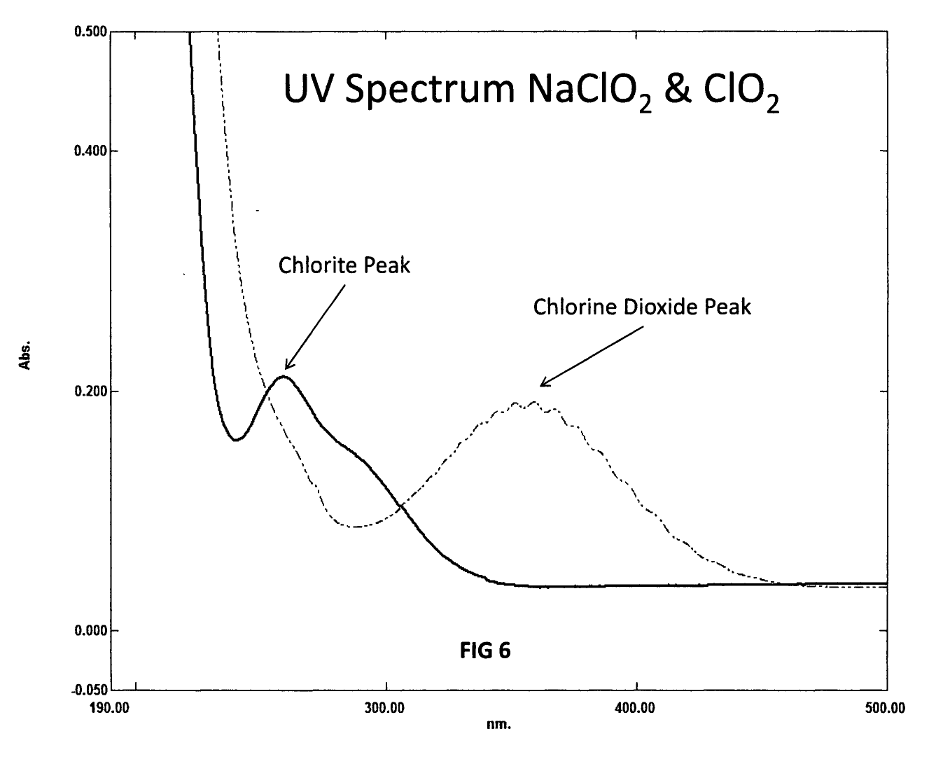

FIG. 6—illustrates the UV-Vis spectrum and characteristic peak of sodium chlorite and chlorine dioxide in distilled water.

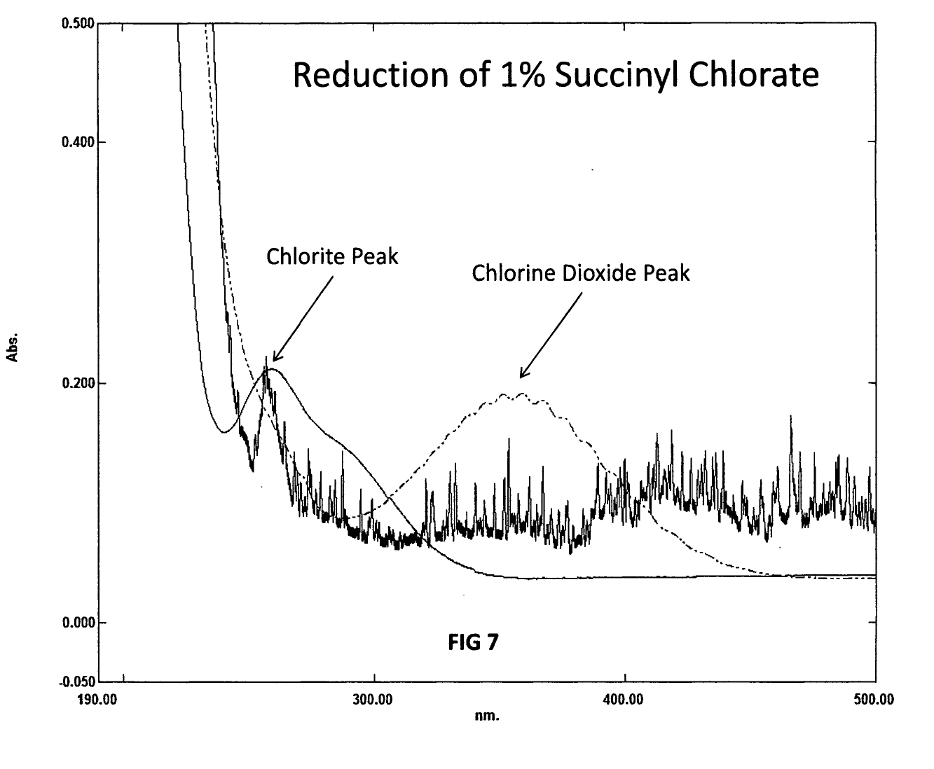

FIG. 7—illustrates the UV-Vis spectrum of succinyl chlorate undergoing reduction in the presence of organic contaminated ceramic cylinders, and compares the characteristic peaks of sodium chlorite and chlorine dioxide (FIG. 6) to those produced during the cascading decomposition of succinyl chlorate. Distinct peaks at both the chlorine dioxide and chlorite wavelengths illustrate the presence of the oxychlorine intermediates resulting from reduction of the acyl polyoxychlorine.

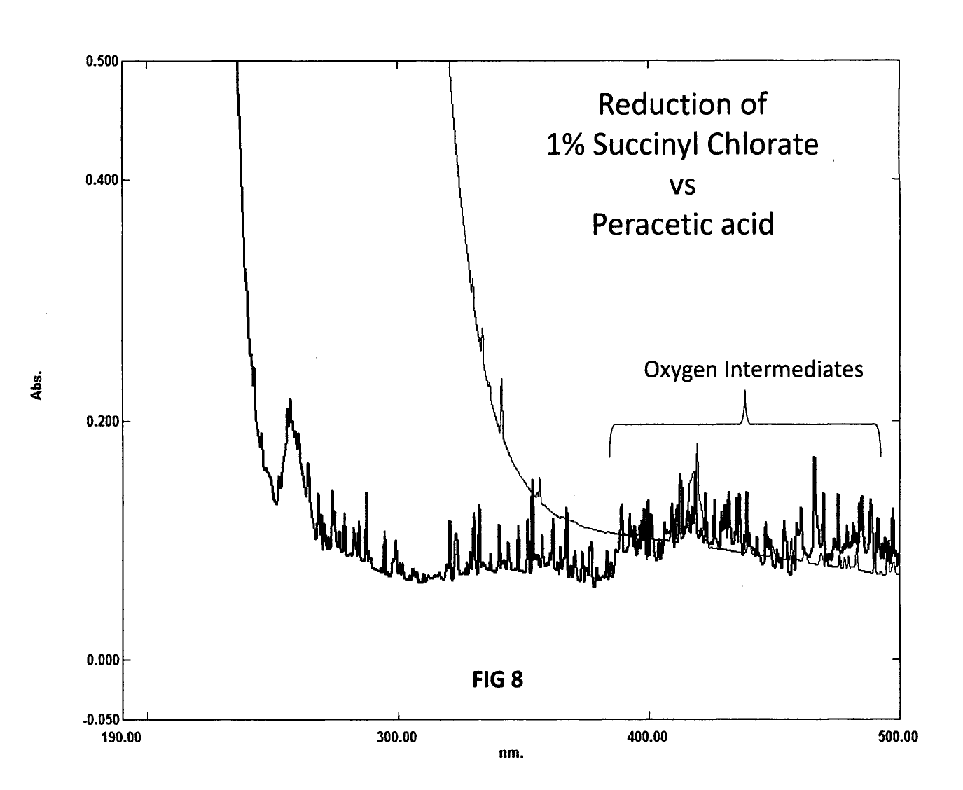

FIG. 8—illustrates a comparison between the UV-Vis spectrum resulting from reduction of succinyl chlorate and reduction of peracetic acid. Almost a perfect overlay occurs at approximately 420 nm, however succinyl chlorate demonstrates higher magnitude peaks over a much broader region of the UV-Vis spectrum. The distinct peaks (associated with oxygen species) illustrate the release of oxygen species during the cascading decomposition of the oxychlorine intermediates.

### OAP Based Antibiotic Function

When the OAP based antibiotic enters the cell wall of bacteria, the β-Lactam group will be activated by enzymes exemplified by DD-transpeptidase and β-Lactamase. When the β-Lactam ring is cleaved, the “R¹” group (“OAP leaving group”) is released from the cephalosporin carrier as illustrated (see FIG. 3).

The released OAP leaving group is now unhindered by the steric hindrance provided by the cephalosporin carrier, and is available to perform its biocidal function.

Without being bound to a specific theory, it is proposed there may be two mechanisms that provide the biocidal efficacy of the OAP (reference FIG. 3B).

In the non-limiting example designated option 1, the OAP leaving group remains intact and functions autonomously within the cell wall. The released OAP can then permeate through the final membrane (plasma membrane) of the bacteria for maximum biocide efficiency. In one example, it is theorized the O-acyl carbon bond is cleaved by enzymatic attack exemplified by “Acetyl coenzyme A”, releasing an unstable oxychlorine intermediate. The unstable oxychlorine intermediate undergoes cascading decomposition within the cytoplasm of the bacteria, thereby inducing catastrophic failure and cell death.

Permeation through the final cell membrane (e.g. plasma membrane) may be further enhanced by incorporating substituted groups onto the carbon based backbone thereby altering its octanol/water partition and/or charge distribution to improve permeation through the plasma membrane.

In the non-limiting example designated option 2, the polyoxychlorine group is reduced either within the cell wall or once it has permeated the plasma membrane, liberating a carboxyl group and an oxychlorine intermediate (e.g. chlorine dioxide). In this example, the chlorine dioxide is the initial oxychlorine intermediate that undergoes cascading decomposition, eventually terminating as oxygen and chloride.

The initial reduction resulting in the release of chlorine dioxide may result from the OAP acquiring the electron withdrawn from the β-Lactam group released by enzymatic attack within the cell wall. Another possible path for initial reduction may be direct reaction between the OAP and chemical reductants within the cell wall or cytoplasm. Regardless of the exact mechanisms, the release of oxychlorine intermediates within the bacteria induces oxidative stress and cell death.

### OAP Based Antibiotic Benefits

The benefits of using OAP based antibiotics include but may not be limited to:

1) Utilizing β-Lactam based antibiotic provides the ability to selectively target bacteria within mammals since the β-Lactam group is activated by enzymes found only within the cell wall of bacteria.
2) Low concentrations and/or shorter periods of use of OAP based antibiotics may be used due to the high lethality of the OAP. Rather than having to accumulate high concentrations of antibiotic within the cell wall to inhibit construction of the cell wall and/or overcome the effects of β-Lactamase, only enough OAP based antibiotic is necessary to deliver a few molecules of OAP leaving group into the cell wall and/or cytoplasm to cause catastrophic oxidative stress.
3) The OAP based antibiotic achieves efficient kills regardless of whether the β-Lactam group is activated by DD-transpeptidase or β-Lactamase. In effect, the antibiotic efficacy of the cephalosporin carrier becomes a mute function. The ability of the cephalosporin carrier to selectively target and release the OAP leaving group becomes the primary function.
4) The cephalosporin carrier imparts steric hindrance thereby protecting the polyoxychlorine group from premature reduction while passing through the metabolic pathways to the target bacteria.
5) OAP based antibiotics can eliminate the potential for bacteria mutation and creation of a Super-Bug. Resistant strains which utilize β-Lactamase (MRSA) can efficiently be inactivated.
6) The potential for bacteria to develop a resistance to OAP based antibiotics is highly unlikely. Common bacteria have been exposed to chlorine for over 100 years without mutating into resistant forms against chlorine. Until the present invention, there has not been an efficient and pharmaceutically acceptable means for introducing oxychlorine into the bacteria.

### OAP Based Antibiotic Toxicity

Design of the OAP based antibiotic will help ensure passage through the body with minimal toxicity due to premature decomposition of the polyoxychlorine group and localized oxidative stress. As long as the polyoxychlorine group is prevented from premature reduction, there is minimal potential of toxicity. The OAP leaving group becomes a functional biocide only after the β-Lactam group has been activated and the OAP leaving group is released from the carrier cephalosporin. To further improve upon survivability and undesired activation, it is reasonable to envision the organic acyl polyoxychlorine compound “caged” within a structure, or sufficiently sheltered by incorporating substitution groups onto the carbon based backbone.

Only low concentrations (low MIC) would now be required due to the efficiency and lethality of the OAP based antibiotic. Little excess antibiotic would be required and would therefore impart low potential for renal toxicity due to oxidative stress.

Short-use-duration would be expected since there would be no need to accumulate the antibiotic within the cell wall. Only low concentrations are needed to effectively inactivate the bacteria thereby substantially increasing the rate of inactivation and requiring subsequent shortened duration of use.

To further offset the potential for residual antibiotic inducing oxidative stress within the renal system, OAP based antibiotic formulations could comprise Nephroprotective agents exemplified by the non-limiting examples N-Acetyl Cysteine (NAC). If the OAP leaving group is decomposed, the potential for oxidative stress could be minimized by reduction of the reactive intermediates with NAC, and/or increased glutathione levels in the cells resulting from the presence of the glutathione precursor NAC. Other Nephroprotective agents may include reducing agents including the non-limiting examples comprising glutathione, ascorbic acid, selective amino acids exemplified by glycine, and compounds comprising thiol based groups.

It is conceivable to incorporate a Nephroprotective agent onto the “R²” group of the cephalosporin to serve a dual function in the event of premature reduction of the polyoxychlorine group.

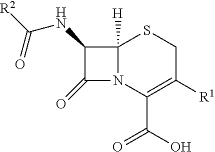

Since the outer membrane of gram negative bacteria favor passage of hydrophilic compounds, incorporating a hydrophilic group such as NAC at the R² site could enhance permeation into gram negative bacteria as well as provide localized neutralization of oxychlorine and oxygen reactive intermediates should decomposition of the OAP leaving group occur in the renal system.

Since the OAP leaving group would still be attached to the host carrier cephalosporin while passing through the renal system, the neutralizing agents at the R² group would be locally available to interact with the free radicals produced thereby minimizing oxidative stress and cell damage. Nephroprotective agents can also be synthesized onto other OAP based antibiotics that do not comprise cephalosporin.

While the preferred method of delivery of the organic acyl polyoxychlorine is by designing the OAP based antibiotic with the OAP leaving group that is released when the β-Lactam group is reacted, activation of the OAP leaving group within the cell wall would also impart catastrophic failure of the cell wall and induce lysis leading to cell death. Therefore, it can also be beneficial to bond the OAP leaving group to structures comprising β-Lactam wherein the OAP leaving group is not released from the β-Lactam carrier. Furthermore, the β-Lactam carrier does not have to possess antibiotic activity. As long as the β-Lactam carrier reacts with DD-transpeptidase and/or β-Lactamase within the bacteria cell wall, the OAP leaving group is being delivered into the bacteria cell. Other enzyme or metabolic actions could activate the OAP leaving group leading to cell death. Therefore the bacteria activated Beta-Lactam group does not have to be antibiotic.

Furthermore, β-Lactamase inhibitors exemplified by the non-limiting examples sulbactam, claulanic acid, and tazobactam, may be synthesized to include organic acyl polyoxychlorine functionality.

The OAP based antibiotics of the invention can be formulated to be administered to mammals in a variety of convenient ways. Some non-limiting example of how OAP based antibiotic can be administered include: intravenous, oral, topical, nasal spray, drops, and the like.

The OAP based antibiotics of the invention can be formulated with pharmaceutically accepted carriers or diluents that are selected based on the method of delivery. Some non-limiting examples may include: pregelatinized starch, maltodextrin, isomalt, sobitol syrup, mannitol, erythritol, maize starch, nanocarriers such as liposomes, micelles, polymeric nanoparticles, and peptides.

I claim:

1. An antibiotic composition for killing bacteria comprising: an organic acyl polyoxychlorine coupled to a β-Lactam carrier.

2. The composition according to claim 1, wherein the organic acyl polyoxychlorine having the general formula: 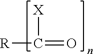
wherein (X) comprises a polyoxychlorine;
(R) comprises a carbon based backbone coupled to an atom directly appending the acyl carbon;
where (n) is one or more.

3. The composition according to claim 2, wherein the polyoxychlorine comprises chlorate having the general formula ClO₃.

4. The composition according to claim 2, wherein the polyoxychlorine comprises perchlorate having the general formula ClO₄.

5. The composition according to claim 2, wherein an atom directly appending the acyl carbon comprises carbon.

6. The composition according to claim 2, wherein an atom directly appending the acyl carbon comprises nitrogen.

7. The composition according to claim 2, wherein an atom directly appending the acyl carbon comprises oxygen.

8. The composition according to claim 2, wherein an atom directly appending the acyl carbon comprises sulfur.

9. The composition according to claim 2, wherein an atom directly appending the acyl carbon comprises phosphorous.

10. The composition according to claim 1, wherein the β-Lactam carrier comprises cepholasporin.

11. The composition according to claim 1, wherein the β-Lactam carrier comprises carbapenem.

12. The composition according to claim 1, wherein the β-Lactam carrier is activated by beta-lactamase.

13. The composition according to claim 1, wherein the β-Lactam carrier is activated by DD-transpeptidase.

14. The composition according to claim 1, wherein the β-Lactam carrier is not an antibiotic.

15. The composition according to claim 1, further comprising a pharmaceutically acceptable carrier.

16. The composition according to claim 1, wherein the antibiotic is effective against Methicillin-resistant Staphylococcus aureus.

17. A method for killing bacteria comprising contacting the bacteria with the antibiotic composition according to claim 1.

18. The method for killing bacteria according to claim 17, the method comprising the steps of: reacting a polyoxychlorine donor with an organic acyl donor to form an OAP leaving group; coupling the OAP leaving group and a Beta-lactam carrier to form an OAP based antibiotic; combining said OAP based antibiotic and a pharmaceutically acceptable carrier to form an antibiotic composition; administering said antibiotic composition to a mammal; absorbing the OAP based antibiotic into the peptidoglycan cell wall of bacteria; contacting the OAP based antibiotic with an enzyme to activate the Beta-Lactam group; releasing of the OAP leaving group, and reduction of the organic acyl polyoxychlorine releases oxychlorine intermediates that induce oxidative stress and bacteria death.

19. The method for killing bacteria according to claim 17, the method comprising the steps of: reacting a polyoxychlorine donor with an organic acyl donor to form an OAP leaving group; coupling the OAP leaving group and Beta-lactam carrier to form an OAP based antibiotic; combining an OAP based antibiotic and a pharmaceutically acceptable carrier to form an antibiotic composition; administering said antibiotic composition to a mammal; absorbing the OAP based antibiotic into the peptidoglycan cell wall of bacteria; contacting the OAP based antibiotic with an enzyme to activate the Beta-Lactam group; release of oxychlorine intermediates, and reduction of the oxychlorine intermediates induces oxidative stress and bacteria death.
